(19) United States
(12) Patent Application Publication
(10) Pub. No.: US 2019/0381058 A1
(43) Pub. Date: Dec. 19, 2019

(54) METHODS AND COMPOSITIONS FOR TREATING NEUROBLASTOMA IN A JUVENILE MAMMALIAN BODY

(71) Applicant: CorMedix Inc., Berkeley Heights, NJ (US)

(72) Inventors: Bruce Reidenberg, Rye, NY (US); Robert DiLuccio, Viera, FL (US)

(21) Appl. No.: 16/554,100

(22) Filed: Aug. 28, 2019

Related U.S. Application Data
(63) Continuation-in-part of application No. 15/403,876, filed on Jan. 11, 2017.
(60) Provisional application No. 62/277,243, filed on Jan. 11, 2016.
(60) Provisional application No. 62/723,592, filed on Aug. 28, 2018.

(51) Int. Cl. A61K 31/549; A61K 9/14; A61K 9/00

(52) U.S. Cl. CPC A61K 31/549; A61K 9/0019; A61K 9/14

(57) ABSTRACT

Neuroblastoma is a tumor primarily affecting children. The current standard of care is not curative except in the rare case of a surgically-resectable lesion, although very high survival rates have been documented for low-risk neuroblastoma and moderate-risk neuroblastoma. Taurolidine was developed as an anti-infective, but it has been found to have surprising oncolytic activity in cell cultures and now in a rodent cancer model. This invention relates to the use of taurolidine for the treatment of neuroblastoma in juvenile mammals.

### REFERENCE TO PENDING PRIOR PATENT APPLICATIONS

This patent application:

(i) is a continuation-in-part of pending prior U.S. patent application Ser. No. 15/403,876, filed Jan. 11, 2017 by CorMedix Inc. and Robert DiLuccio for THERAPEUTIC NANOPARTICLES FOR THE TREATMENT OF NEUROBLASTOMA AND OTHER CANCERS (Attorney's Docket No. CORMEDIX-14), which patent application claims benefit of prior U.S. Provisional Patent Application Ser. No. 62/277,243, filed Jan. 11, 2016 by CorMedix Inc. and Robert DiLuccio for NANOPARTICLE SYSTEM FOR THE TREATMENT OF NEUROBLASTOMA (Attorney's Docket No. CORMEDIX-14 PROV); and

(ii) claims benefit of pending prior U.S. Provisional Patent Application Ser. No. 62/723,592, filed Aug. 28, 2018 by CorMedix Inc. and Bruce Reidenberg et al. for METHODS AND COMPOSITIONS FOR TREATING NEUROBLASTOMA IN A JUVENILE MAMMALIAN BODY (Attorney's Docket No. CORMEDIX-32 PROV).

The three (3) above-identified patent applications are hereby incorporated herein by reference.

### FIELD OF THE INVENTION

This invention relates to therapeutic methods and compositions in general, and more particularly to therapeutic methods and compositions for the treatment of neuroblastoma in a juvenile mammalian body.

### BACKGROUND OF THE INVENTION

Neuroblastoma (NB) is the most common extracranial solid cancer in childhood, and the most common cancer in infancy, with an incidence of about six hundred fifty cases per year in the U.S., and a hundred cases per year in the UK. Nearly half of neuroblastoma cases occur in children younger than two years. It is a neuroendocrine tumor, arising from any neural crest element of the sympathetic nervous system (SNS). Neuroblastoma most frequently originates in one of the adrenal glands, but can also develop in nerve tissues in the neck, chest, abdomen, or pelvis. Note that while neuroblastoma arises from nerve tissues, it is not a tumor of the central nervous system (CNS).

Neuroblastoma is one of the few human malignancies known to demonstrate spontaneous regression from an undifferentiated state to a completely benign cellular appearance.

Neuroblastoma is a disease exhibiting extreme heterogeneity, and is stratified into three risk categories: low-risk, intermediate-risk, and high-risk. Low-risk neuroblastoma is most common in infants and good outcomes are common with observation only or surgery, whereas high-risk neuroblastoma is difficult to treat successfully even with the most intensive multi-modal therapies available.

When the neuroblastoma lesion is localized, it is generally curable. However, long-term survival for children older than 18 months of age with advanced disease is poor, despite aggressive multimodal therapy, e.g., intensive chemotherapy, surgery, radiation therapy, stem cell transplant, differentiation agent isotrentinoin (also called 13-cis-retinoic acid), and frequently immunotherapy with anti-GD2 immunotherapy with anti-GD2 monoclonal antibody therapy.

Biologic and genetic characteristics have been identified which, when added to classic clinical staging, has allowed patient assignment to risk groups for planning treatment intensity. These criteria include age of the patient, extent of disease spread, microscopic appearance, and genetic features including DNA ploidy and N-myc oncogene amplification (N-myc regulate micro RNAs). These criteria are used to classify the neuroblastoma into low-risk, intermediate-risk, and high-risk disease. A recent biology study (COG ANBL00B1) analyzed 2,687 neuroblastoma patients and the spectrum of risk assignment was determined: 37% of neuroblastoma cases are low-risk, 18% of neuroblastoma cases are intermediate-risk, and 45% of neuroblastoma cases are high-risk. Note that there is some evidence that the high-risk and low-risk types of neuroblastoma are caused by different mechanisms, and are not merely two different degrees of expression of the same mechanism.

The therapies for these different risk categories are very different.

Low-risk neuroblastoma can frequently be observed without any treatments at all or cured with surgery alone.

Intermediate-risk neuroblastoma is generally treated with surgery and chemotherapy.

High-risk neuroblastoma is generally treated with intensive chemotherapy, surgery, radiation therapy, bone marrow/hematopoietic stem cell transplantation, biological-based therapy with 13-cis-retinoic acid (isotretinoin or Accutane) and antibody therapy (usually administered with the cytokines GM-CSF and IL-2. cytokines).

With current treatments, patients with low-risk neuroblastoma and intermediate-risk neuroblastoma have an excellent prognosis, with cure rates above 90% for low-risk neuroblastoma and 70-90% cure rates for intermediate-risk neuroblastoma. In contrast, therapy for high-risk neuroblastoma over the past two decades has resulted in cures only about 30% of the time. The addition of antibody therapy has raised survival rates for high-risk neuroblastoma significantly. In March 2009, an early analysis of a Children's Oncology Group (COG) study with 226 high-risk neuroblastoma patients showed that two years after stem cell transplant, 66% of the group randomized to receive ch14.18 antibody with GM-CSF and IL-2 were alive and disease-free, compared to only 46% in the group that did not receive the antibody. The randomization was stopped so all patients enrolling in the trial could receive the antibody therapy.

Chemotherapy agents used in combination have been found to be effective against neuroblastoma. Agents commonly used in induction and for stem cell transplant conditioning are platinum compounds (cisplatin, carboplatin), alkylating agents (cyclophosphamide, ifosfamide, melphalan, topoisomerase II inhibitor) and vinca alkaloids (vincristine). Some newer regimens include topoisomerase I inhibitors (topotecan and irinotecan) in induction which have been found to be effective against recurrent disease.

However, a need exists for a new method and composition which are effective against neuroblastoma in a juvenile mammalian body.

### SUMMARY OF THE INVENTION

In accordance with the present invention, taurolidine is used to treat neuroblastoma in juvenile mammalian bodies.

The taurolidine is given with a dosage range of from 5 mg/kg to 280 mg/kg, and preferably with a dosage range of between 5 mg/kg and 60 mg/kg.

This dosage is administered from once daily through weekly for an effective period of time based on individual patient response.

The taurolidine is delivered systemically, preferably either intravenously (more preferred) or intramuscularly.

In one preferred form of the invention, the taurolidine is delivered systemically in a “shielded form” so that hydrolysis of the taurolidine is delayed until the taurolidine reaches the site of the neuroblastoma, whereupon hydrolysis of the taurolidine occurs.

The taurolidine may be delivered as a single agent or in combination with one or more oncolytic agents and/or radiotherapy.

In one form of the invention, there is provided a method for treating neuroblastoma in juvenile mammals, the method comprising administering taurolidine to the juvenile mammal.

In one form of the invention, the taurolidine is administered with a dosage range of from 5 mg/kg to 280 mg/kg, for an effective period of time, based on individual patient response.

In one form of the invention, the taurolidine is administered with a dosage range of from 5 mg/kg and 60 mg/kg.

In one form of the invention, the dosage is administered from once daily through weekly.

In one form of the invention, the taurolidine is administered systemically.

In one form of the invention, the taurolidine is administered intravenously.

In one form of the invention, the taurolidine is administered intramuscularly.

In one form of the invention, the taurolidine is included in a nanoparticle, and the nanoparticle is configured to delay hydrolysis of the taurolidine until the nanoparticle reaches the site of a tumor.

In one form of the invention, the taurolidine is included in a nanoparticle, the nanoparticle comprises a taurolidine core and an exterior coating, and the exterior coating is configured to prevent exposure of the taurolidine prior to arrival of the nanoparticle at the site of the tumor.

In one form of the invention, the taurolidine is included in a nanoparticle, the nanoparticle comprises a taurolidine core and an exterior coating, and the exterior coating comprises an absorbable polymer or lipid which breaks down as the nanoparticle travels from the site of insertion to the site of the tumor.

In one form of the invention, the taurolidine is delivered using a polymer system which is configured to delay hydrolysis of the taurolidine.

In one form of the invention, the taurolidine is delivered using a polymer system, with the taurolidine being “pegylated” using polyethylene glycols (PEGs) to delay premature of hydrolysis of taurolidine.

In one form of the invention, the taurolidine is administered to humans.

In one form of the invention, the taurolidine is administered to at least one from the group consisting of infants, children and adolescents.

In one form of the invention, the taurolidine is administered as a single agent.

In one form of the invention, the taurolidine is administered in combination with at least one oncolytic agent.

In one form of the invention, the taurolidine is administered in combination with at least one oncolytic agent, and the at least one oncolytic agent is selected from the group consisting of platinum compounds (cisplatin, carboplatin), alkylating agents (cyclophosphamide, ifosfamide, melphalan, topoisomerase II inhibitor), vinca alkaloids (vincristine), and topoisomerase I inhibitors (topotecan and irinotecan).

In one form of the invention, the taurolidine is administered in combination with radiotherapy.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a graph showing that leukemia cell lines appear more sensitive to the effects of taurolidine compared to healthy lymphocytes in vitro (not in vivo);

FIG. 2 is a graph showing that neuroblastoma cell lines are more sensitive to a decrease in viability due to taurolidine when compared to healthy fibroblasts (BJ on graph) in vitro (not in vivo);

FIGS. 3-6 are graphs or photographs showing that taurolidine given to CB57 SCID mice with measurable tumors from a neuroblastoma cell line implanted subcutaneously in the CB57 SCID mice has efficacy in IMR5 tumors and measurable efficacy in SK-N-AS tumors in vivo (not in vitro); and

FIGS. 7 and 8 are graphs showing that statistically significant decreases in tumor size were achieved when taurolidine was administered to treat mice with a different cell line (SK-N-AS) also derived from neuroblastoma but overall survival was not significantly different from control.

### DETAILED DESCRIPTION OF THE INVENTION

Taurolidine is a well known antimicrobial with a published mechanism of action and antimicrobial spectrum. Taurolidine is unstable in circulation and therefore has not been successfully developed for systemic infections. Taurolidine has demonstrated efficacy in local application for peritonitis and for prevention of infection when infused as a catheter-lock solution.

Taurolidine has recently been investigated for oncolytic activity and found to have inhibitory effect on cell lines in culture, in combination with standard chemotherapy or alone. Despite claims that in vitro inhibitory concentrations are clinically achievable, the only published human pharmacokinetic study showed NO measurable concentration of taurolidine in healthy volunteers when 5 grams of taurolidine were given intravenously by 20 minute infusion. This is believed to be due to the rapid hydrolysis of taurolidine when administered systemically in a mammalian body.

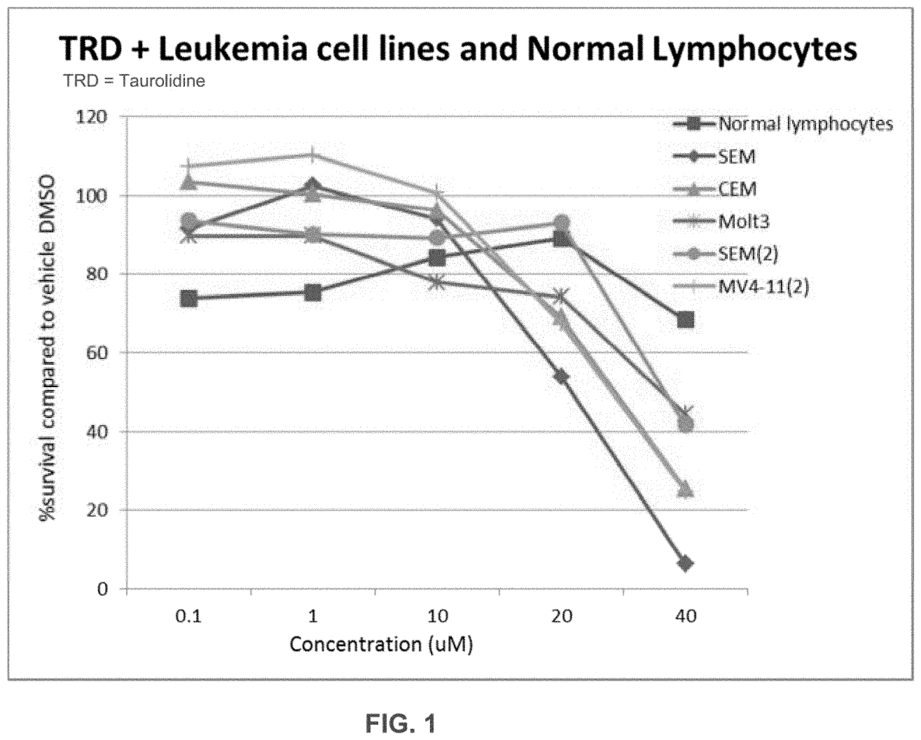

It has been found that leukemia cell lines appear more sensitive to the effects of taurolidine compared to healthy lymphocytes in vitro (not in vivo). See FIG. 1.

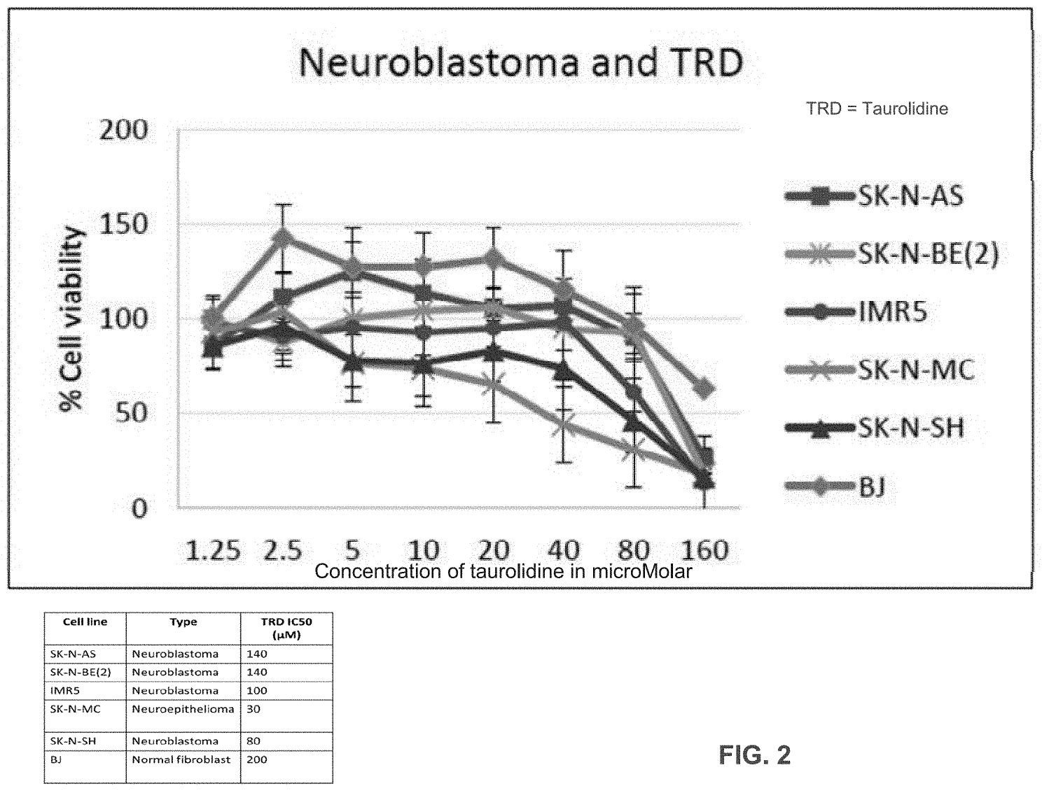

It has also been found that neuroblastoma cell lines are more sensitive to a decrease in viability due to taurolidine when compared to healthy fibroblasts in vitro (not in vivo). See FIG. 2.

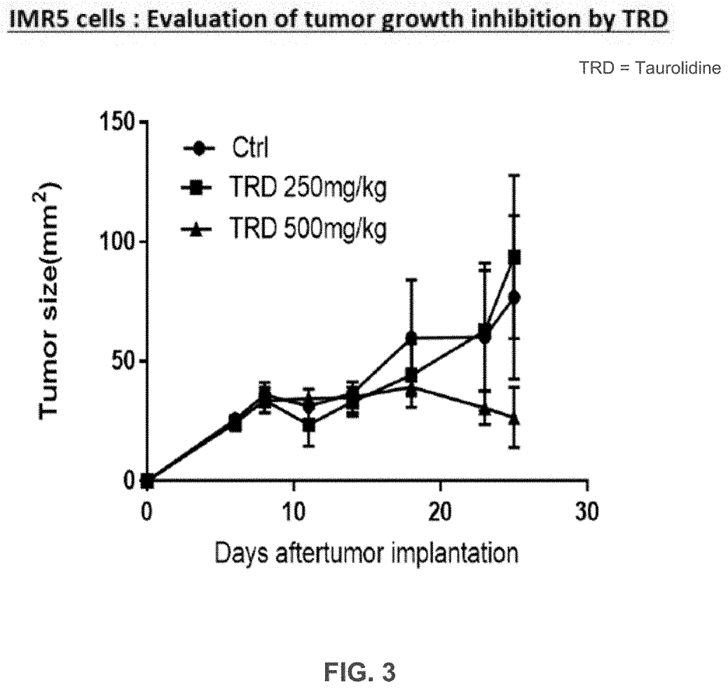

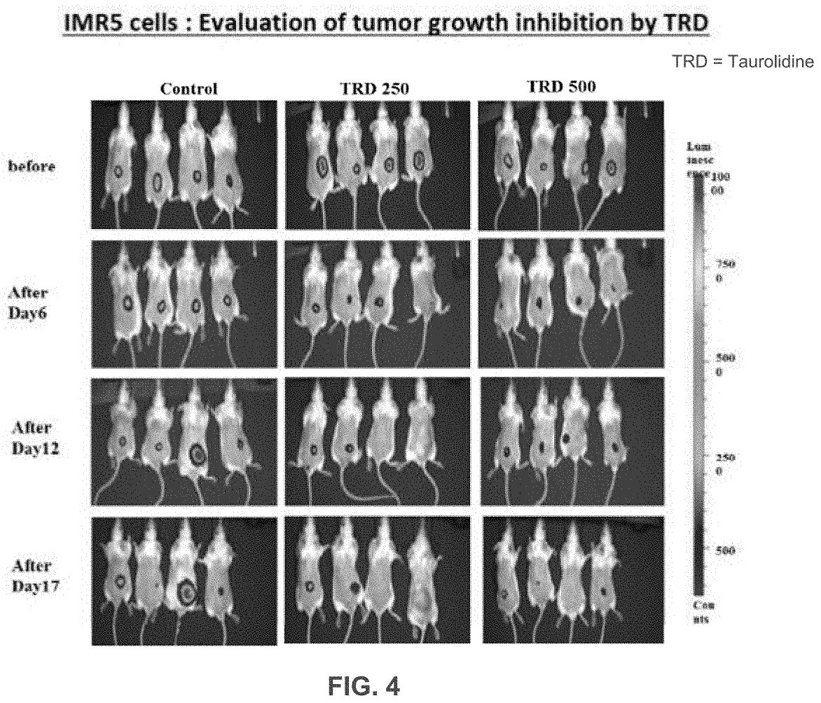

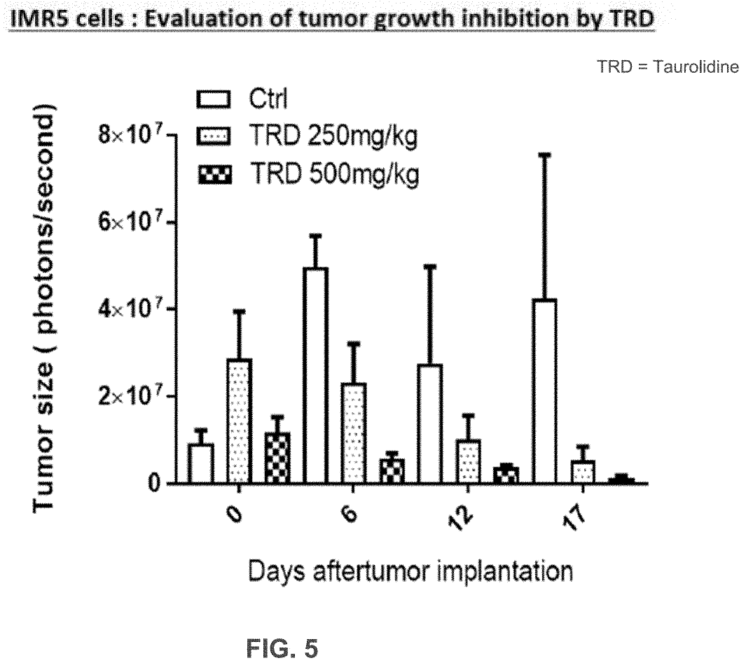

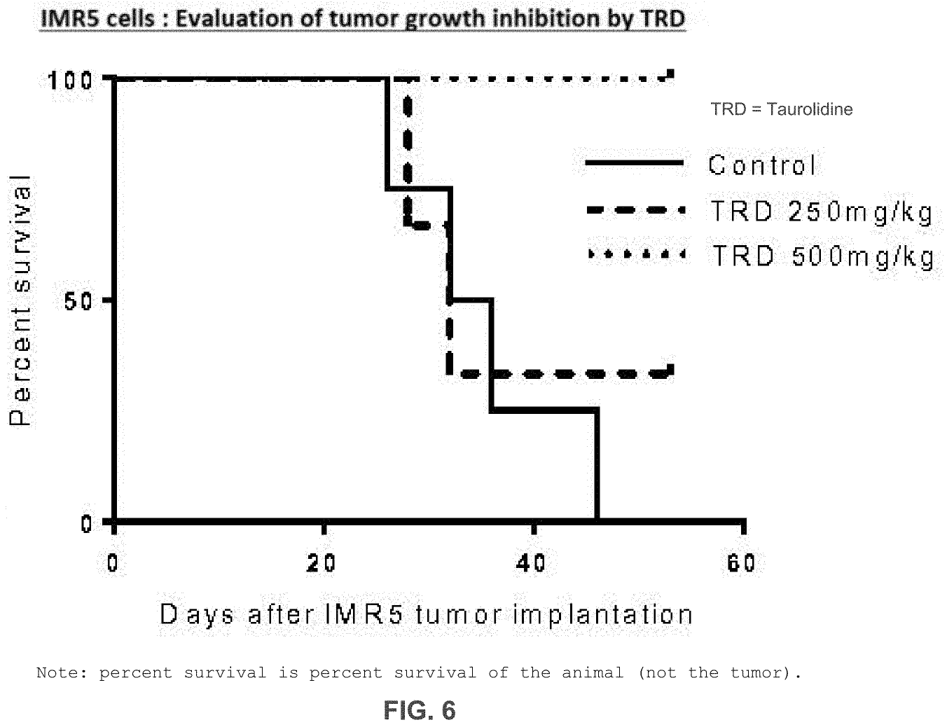

Furthermore, taurolidine given to CB57 SCID mice with measurable tumors from a neuroblastoma cell line implanted subcutaneously in the CB57 SCID mice showed efficacy in IMR5 tumors and measurable efficacy in SK-N-AS tumors in vivo (not in vitro). See FIGS. 3-6.

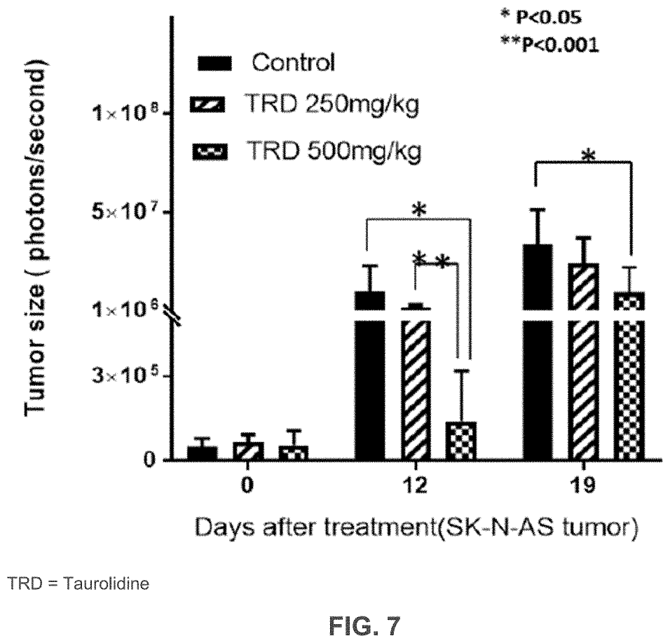

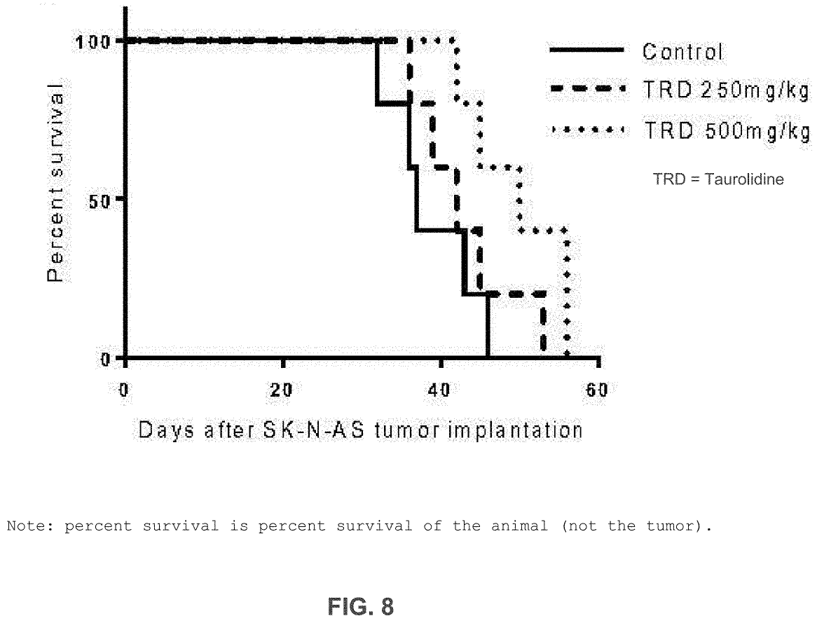

Statistically significant decreases in tumor size were achieved when taurolidine was administered to treat mice with a different cell line (SK-N-AS) also derived from neuroblastoma, though overall survival of the mice implanted with the tumor was not statistically different from the control. See FIGS. 7 and 8.

It has now been discovered that taurolidine may be used to treat neuroblastoma in a juvenile mammalian body.

The taurolidine is given with a dosage range of from 5 mg/kg to 280 mg/kg, and preferably with a dosage range of between 5 mg/kg and 60 mg/kg. Effective dosage was computed by computing the human equivalent dosage from the effective mouse dose, using the following formula:

Human equivalent dose=mouse mg/kg dose×1 adult human/12 mice×25 child BSA ratio/37 adult BSA ratio=child dose in mg/kg

(https://www.fda.gov/downloads/drugs/guidances/ucm0789 32.pdf).

This dosage is administered from once daily through weekly for an effective period of time based on individual patient response.

The taurolidine is delivered systemically, preferably either intravenously (more preferred) or intramuscularly. In one preferred form of the invention, the taurolidine is delivered systemically in a “shielded form” so that hydrolysis of the taurolidine is delayed until the taurolidine reaches the site of the neuroblastoma, whereupon hydrolysis of the taurolidine occurs.

More particularly, in one preferred form of the invention, the taurolidine is delivered in the form of a nanoparticle, where the nanoparticle comprises a taurolidine core and an exterior coating which is configured to prevent premature exposure of the taurolidine prior to the arrival of the nanoparticle to the tumor site. The exterior coating breaks down as the nanoparticle travels from the site of insertion to the site of the tumor so as to release the taurolidine for hydrolysis at the site of the tumor. In one preferred form of the invention, the coating comprises an absorbable polymer or lipid which breaks down as the nanoparticle travels from the site of insertion to the site of the tumor. By way of example but not limitation, the coating can be created from combinations of copolymers and multimers derived from polymers structured from 1-lactide, glycolide, e-caprolactone, p-dioxanone, and trimethylene carbonate. The coating may also be associated with glycols such as polyethylene glycols (PEGs), which can either be linear or multi-arm structures.

If desired, the nanoparticle may comprise an excipient (e.g., a buffer for providing enhanced hydrolytic stability of the taurolidine within the nanoparticle).

Additionally, if desired, the nanoparticle can further comprise a coating, wherein the coating is configured to target the nanoparticle to the site of a neuroblastoma so as to improve the efficacy of the taurolidine for treatment of the neuroblastoma. In one preferred form of the invention, the coating comprises binding molecules which are configured to target delivery of the nanoparticle to specific tissue. By way of example but not limitation, the coating for the nanoparticle comprises a monoclonal antibody against N-type calcium channels (e.g., an anti-N-type calcium channel exofacial Fab fragment) for causing the nanoparticle to bind to neural tissue (e.g., to a neuroblastoma tumor).

In another form of the invention, the taurolidine may be delivered using a polymer system which is configured to delay hydrolysis of the taurolidine and/or optimize the release properties of the taurolidine. By way of example but not limitation, the taurolidine may be “pegylated” using polyethylene glycols (PEGs) to delay premature of hydrolysis of taurolidine and/or optimize the release properties of the taurolidine.

The taurolidine may be delivered as a single agent or in combination with one or more oncolytic agents and/or radiotherapy. Examples of oncolytic agents that can be combined with taurolidine for delivery to a juvenile mammal for treating neuroblastoma are platinum compounds (cisplatin, carboplatin), alkylating agents (cyclophosphamide, ifosfamide, melphalan, topoisomerase II inhibitor), vinca alkaloids (vincristine), and topoisomerase I inhibitors (topotecan and irinotecan).

### MODIFICATIONS

While the present invention has been described in terms of certain exemplary preferred embodiments, it will be readily understood and appreciated by those skilled in the art that it is not so limited, and that many additions, deletions and modifications may be made to the preferred embodiments discussed above while remaining within the scope of the present invention.

What is claimed is:

1. A method for treating neuroblastoma in juvenile mammals, the method comprising administering taurolidine to the juvenile mammal.

2. A method according to claim 1 wherein the taurolidine is administered with a dosage range of from 5 mg/kg to 280 mg/kg, for an effective period of time, based on individual patient response.

3. A method according to claim 2 wherein the dosage range is from 5 mg/kg and 60 mg/kg.

4. A method according to claim 1 wherein the dosage is administered from once daily through weekly.

5. A method according to claim 1 wherein the taurolidine is administered systemically.

6. A method according to claim 5 wherein the taurolidine is administered intravenously.

7. A method according to claim 5 wherein the taurolidine is administered intramuscularly.

8. A method according to claim 5 wherein the taurolidine is included in a nanoparticle, and further wherein the nanoparticle is configured to delay hydrolysis of the taurolidine until the nanoparticle reaches the site of a tumor.

9. A method according to claim 8 wherein the nanoparticle comprises a taurolidine core and an exterior coating, wherein the exterior coating is configured to prevent exposure of the taurolidine prior to arrival of the nanoparticle at the site of the tumor.

10. A method according to claim 9 wherein the exterior coating comprises an absorbable polymer or lipid which breaks down as the nanoparticle travels from the site of insertion to the site of the tumor.

11. A method according to claim 5 wherein the taurolidine is delivered using a polymer system which is configured to delay hydrolysis of the taurolidine.

12. A method according to claim 11 wherein the taurolidine is “pegylated” using polyethylene glycols (PEGs) to delay premature of hydrolysis of taurolidine.

13. A method according to claim 1 wherein the taurolidine is administered to humans.

14. A method according to claim 13 wherein the taurolidine is administered to at least one from the group consisting of infants, children and adolescents.

15. A method according to claim 1 wherein the taurolidine is administered as a single agent.

16. A method according to claim 1 wherein the taurolidine is administered in combination with at least one oncolytic agent.

17. A method according to claim 16 wherein the at least one oncolytic agent is selected from the group consisting of platinum compounds (cisplatin, carboplatin), alkylating agents (cyclophosphamide, ifosfamide, melphalan, topoisomerase II inhibitor), vinca alkaloids (vincristine), and topoisomerase I inhibitors (topotecan and irinotecan).

18. A method according to claim 1 wherein the taurolidine is administered in combination with radiotherapy.
